(19) United States
(12) Patent Application Publication
(10) Pub. No.: US 2015/0329609 A1
(43) Pub. Date: Nov. 19, 2015

(54) CHIMERIC DYSTROPHIN PROTEINS TO TREAT DYSTROPHINOPATHIES

(71) Applicant: Sujata ACHARJEE, New York, NY (US)

(72) Inventor: Sujata ACHARJEE, New York, NY (US)

(21) Appl. No.: 14/744,922

(22) Filed: Jun. 19, 2015

Related U.S. Application Data
(63) Continuation-in-part of application No. 14/635,012, filed on Mar. 2, 2015.
(60) Provisional application No. 62/014,191, filed on Jun. 19, 2014.
(60) Provisional application No. 61/946,961, filed on Mar. 3, 2014.

(51) Int. Cl. C07K 14/47; C12N 7/00; C07K 14/005

(52) U.S. Cl. CPC C07K 14/47; C07K 14/005; C12N 7/00; C12N 2760/20233; A61K 38/00

(57) ABSTRACT

A chimeric protein that is a fusion construct of a series of functional domains is used to deliver a therapeutic agent to a human subject suffering from disease. In some embodiments, the chimeric protein includes a therapeutic region and a transportation region. The transportation region allows the chimeric protein to be moved across a cellular membrane of an affected cell within the subject. The therapeutic region can be effective in the treatment of, for example, muscular dystrophy, diastrophic dysplasia, malignant melanoma, porphyria, alpha-1 antitrypsin deficiency, Aicardi-Goutieres syndrome, cystic fibrosis, progeria, Marfan syndrome, tuberous sclerosis, adrenoleukodystrophy, and the like.

### CROSS-REFERENCE TO RELATED APPLICATIONS

This patent application is a continuation-in-part patent application of U.S. patent application Ser. No. 14/635,012, filed Mar. 2, 2015, which claims the benefit of U.S. Provisional patent application No. 61/946,961, filed Mar. 3, 2014. This patent application also claims a benefit to the filing date of U.S. Provisional patent application No. 62/014,191, filed Jun. 19, 2014. The contents of each of the above-identified patent applications are incorporated herein by reference in their entireties.

### FIELD OF THE DISCLOSURE

Disclosed is a chimeric or fusion protein including a therapeutic construct that is designed for use in the treatment of human subjects suffering from diseases such as muscular dystrophy, diastrophic dysplasia, malignant melanoma, porphyria, alpha-1 antitrypsin deficiency, Aicardi-Goutieres syndrome, cystic fibrosis, progeria, Marfan syndrome, tuberous sclerosis, adrenoleukodystrophy, and the like.

### BACKGROUND OF THE DISCLOSURE

The Dystrophin Glycoprotein complex (DGC) is a very crucial structural component of skeletal and cardiac muscles. It is comprised of dystrophin and a plurality of proteins associated with it and imparts structural stability to the muscle membrane. The physical interactions between the proteins of the DGC form the basis for mechanical linking of the outside of the membrane to the inside and play an important role in mediating biological signaling process. These proteins form an intricate network which stabilizes the membrane as it contracts and relaxes. These interactions are essential in maintaining the structural integrity of the muscle membrane. Lack of any of these components owing to mutation compromises the structural stability leading to muscle damage.

Dystrophin was originally identified through its deficiency in the lethal neuromuscular disorder, Duchenne Muscular Dystrophy (DMD). Skeletal and cardiac muscles that lack functional full-length dystrophin protein are extremely susceptible to tear and damage from the contraction-relaxation activity. In the heart, aortic banding experiments performed on the dystrophin-deficient mdx mouse similarly result in accelerated cardiac damage. These studies demonstrated the essential role of dystrophin and the DGC in protecting the plasma membrane against contraction-induced damage.

The absence of dystrophin in DMD patients leaves the muscle membrane fragile and susceptible to damage upon contraction, leading to destruction of the DGC with loss of mechanical stability and proper mechano-transduction signaling. The dystrophin deficient myofibers undergo repeated rounds of contraction mediated injury with consequent myofiber necrosis that ultimately results in the replacement of myofibers by fibrous and fat tissue; a progressive degeneration and failure of regeneration efficiency also occurs owing to the continuous depletion of muscle precursor cells or satellite cells and their incapability to proliferate, multiply, and differentiate.

Dystrophin has four functional domains: a calponin-like actin binding domain at the amino terminal, a central rod domain of 24 spectrin-like repeats, a cysteine-rich region at the carboxy terminal, and an extreme helical carboxy terminal region. The amino terminal actin binding domain is responsible for anchoring dystrophin to cytoskeletal filamentous actin. Within the central rod domain, spectrin repeats 11 through 17 constitute a second site for binding actin. The cysteine rich region interacts with the intracellular portion of the transmembrane protein beta-dystroglycan and anchors dystrophin to the sarcolemma. The extreme carboxyl-terminal mediates its interaction with syntrophins.

The human dystrophin gene is the largest gene characterized so far. It contains 79 exons, several splicing sites and a number of tissue specific promoters that result in a range of transcripts which form amino terminal truncated dystrophin proteins of varying lengths. Dystrophin is a huge gene with an open reading frame that is 11058 nucleotides long, making it a difficult target to work with. The large size of the dystrophin gene is also responsible for its high frequency of spontaneous mutation, with most of the mutations being deletions. The extent of severity caused by these mutations varies depending on the kind of deletion. Where a deletion results in complete absence of dystrophin protein due to disruption of the reading frame of the gene, severe forms of muscular dystrophy or DMD can occur. Deletions which lead to the formation of truncated proteins result in milder forms of muscular dystrophy such as Becker Muscular Dystrophy. One deletion which removes a central part of dystrophin protein encompassing 5,106 base pairs, almost half the coding sequence, has been reported to cause a very mild form of muscular dystrophy with patients being ambulant even at the age of 61.

Dystrophin gene is one of the first genes identified by reverse genetics. DMD is an X-linked muscular dystrophy with an incidence of one in 3500 young males. DMD is one of the most common hereditary diseases known. The onset can be between the age of 3 to 5 yrs. and depending on the severity of the disease the affected males become non-ambulatory by the age of 13. The other clinical features include scoliosis, muscle weakness and damage, muscle hypertrophy, cardiomyopathy, mental retardation and very high serum creatine kinase levels. This disease ultimately causes death between the ages of 15 to 25 years. The most common cause of death in these patients is respiratory or cardiac failure. Tens of thousands of individuals are living with DMD in the United States, Europe, Australia, Canada, Israel, and Japan alone.

DMD has also been reported in a number of animals including mouse, cats and dogs. Mdx mice that have a premature stop codon mutation on exon 23 of the dystrophin gene, leading to a lack of the mature protein, have long been used as an animal model to study the pathogenesis of the disease. The absence of dystrophin results in an acute onset of skeletal muscle necrosis around 3 weeks of post-natal life, followed by an extensive period of degeneration and regeneration until necrosis gradually decreases and a relatively low level is reached in adult mice (3-4 months) with pathology stabilization. However, the pathology is far more benign than in DMD.

Vesicular Stomatitis Viruses have long been known to cause a number of diseases in humans, such as rabies. These viruses enter their hosts by making an envelope of proteins around them also known as VSV-G glycoproteins which facilitate the fusion of viral membrane with the host cell membrane. VSV-G has been widely used as a tool for gene transfer by pseudo typing viral vectors with VSV-G envelope. Recently it has been shown that LDL receptors present in the membrane of mammalian cells serve as a receptor for the VSV-G proteins and port of entry for the vesicular stomatitis viruses. This probably justifies the pantropic nature of vesicular stomatitis viruses as the LDL receptors are present in a wide range of mammalian cells and tissues.

Utrophin is the autosomal homologue (paralogue) of dystrophin. The gene for utrophin located at chromosome 6 in humans, encodes for a protein with 3,433 amino acids with a predicted mass of 395 KD. Interest in the biology of utrophin has been enhanced by a potential role for the protein in novel therapies for Duchenne muscular dystrophy (DMD). Utrophin is structurally related to dystrophin and almost ubiquitously expressed, unlike dystrophin. In developing and regenerating muscles, expression of utrophin precedes dystrophin and utrophin can be detected along the entire muscle sarcolemma. At birth or in adult healthy muscles, utrophin is then replaced by dystrophin in sarcolemma and its expression is confined to the neuromuscular junctions and myotendinous junctions. Studies of mice in which the utrophin gene has been disrupted provided a useful line of evidence to understand the function of utrophin. While the phenotype of mice lacking utrophin appears normal, mice lacking both dystrophin and utrophin exhibit severe phenotype, developing spinal deformity and dying prematurely. In DMD and mdx striated muscle, the distribution of utrophin changes so that it is expressed throughout the sarcolemma and thereby somewhat compensates for the lack of dystrophin.

Cell penetrating peptides (CPPs) are short peptide sequences which are capable of passing through the membrane bilipid layer and transporting molecules across the cell membrane. The CPPs, also known as the protein transduction domains are employed to facilitate the entry and exit of biomolecules like oligonucleotides, siRNA, proteins and liposomes into and from the cells. The transcription transactivating protein (Tat) and penetratin are among the first CPPs to be described and paved the way to the discovery of other naturally occurring CPPs. A minimal peptide sequence from the Tat protein was identified as required for cellular uptake. Penetratin is a 16-merpeptide derived from the third helix of the homeodomain of Antennapedia. Proof-of-concept of the CPPs in vivo application for the delivery of small peptides and large proteins used the chimeric peptide Transportan, derived from the N-terminal fragment of the neuropeptide galanin, linked to mastoparan, a wasp venom peptide was a great breakthrough. Another transferrin receptor (TfR) binding peptide was identified that bound on a distinct site from transferrin binding site and was internalized with the human TfR via endosomal pathway.

DMD therapies that are currently being developed include DNA- and cell-based therapies, as well as drugs which aim to modulate cellular pathways or gene expression. Attempts have been made to restore the expression of full-length functional protein or short truncated protein either via exon-skipping, gene therapy, stem cells, or small molecules to induce read-through of premature stop-codon mutations. Other promising approaches include small molecules or recombinant proteins to enhance the dystrophin surrogate utrophin, and stabilize or reduce degradation of DGC.

Although the approaches previously used are promising, alternative strategies need to be developed because of the limitations of these approaches, e.g. oligonucleotides used for exon-skipping could not be effectively delivered to all the non-skeletal target muscle tissues such as heart; ataluren aimed to induce read-through of premature stop codons in dystrophin gene could only be used for a patient subpopulation exhibiting mutations displaying premature stop codons; ataluren was not potent enough to show any significant effect during clinical trials on patients treated with the drug. Currently there is no treatment available for DMD and current therapies rely in delaying the progression of the disease by clinically using Prednisone and supportive care with a mean life expectancy in the thirties.

What is desired therefore is a simple yet effective system and method for treating patients with DMD. What is also desired is a singular system and method that allows for treatment of multiple types of dystrophinopathy across a plurality of patient types, resulting in increasing or maintaining the structural integrity of the muscle fiber, limiting muscle damage, and improved muscle strength.

### BRIEF SUMMARY

Disclosed herein is a system and method to alleviate the symptoms associated with certain conditions by delivering chimeric proteins in patients exhibiting symptoms of those conditions.

In some embodiments, the present disclosure is directed to a chimeric protein comprising a dystrophin complexed with a transportation region, wherein the transportation region allows for transport of the chimeric protein across a cellular membrane. In some embodiments, the transportation region is a cell penetrating peptide. In some embodiments, the dystrophin is a truncated dystrophin. In some embodiments, the at least one cell penetrating peptide is selected from the group consisting of transcription transactivating protein, penetratin, transportan, transferrin receptor binding peptide, and combinations thereof. In some embodiments, at least one cell penetrating peptide is attached to either the N-terminal or the N- and the C-terminal. In some embodiments, the at least one cell penetrating peptide is complexed at the N-terminal of the dystrophin. In some embodiments, a utrophin is complexed with the dystrophin. In some embodiments, the transportation region is vesicular stomatitis virus G.

In some embodiments, at least a portion of a sequence of the chimeric protein has a nucleotide sequence selected from the group consisting of: SEQ. ID NO.: 13, SEQ. ID NO.: 15, SEQ. ID NO.: 17, SEQ. ID NO.: 19, and pharmaceutical equivalents thereof. In some embodiments, at least a portion of a sequence of the chimeric protein has an amino acid sequence selected from the group consisting of: SEQ. ID NO.: 14, SEQ. ID NO.: 16, SEQ. ID NO.: 18, SEQ. ID NO.: 20, and pharmaceutical equivalents thereof. In some embodiments, at least a portion of a sequence of the chimeric protein has a nucleotide sequence selected from the group consisting of: SEQ. ID NO.: 21, SEQ. ID NO.: 23, SEQ. ID NO.: 25, SEQ. ID NO.: 27, and pharmaceutical equivalents thereof. In some embodiments, at least a portion of a sequence of the chimeric protein has an amino acid sequence selected from the group consisting of: SEQ. ID NO.: 22, SEQ. ID NO.: 24, SEQ. ID NO.: 26, SEQ. ID NO.: 28, and pharmaceutical equivalents thereof.

In some embodiments, at least a portion of a sequence of the chimeric protein has a nucleotide sequence selected from the group consisting of: SEQ. ID NO.: 29, SEQ. ID NO.: 31, SEQ. ID NO.: 33, SEQ. ID NO.: 35, SEQ. ID NO.: 37, and pharmaceutical equivalents thereof. In some embodiments, at least a portion of a sequence of the chimeric protein has an amino acid sequence selected from the group consisting of: SEQ. ID NO.: 30, SEQ. ID NO.: 32, SEQ. ID NO.: 34, SEQ. ID NO.: 36, SEQ. ID NO.: 38, and pharmaceutical equivalents thereof.

In some embodiments, at least a portion of a sequence of the chimeric protein has a nucleotide sequence selected from the group consisting of: SEQ. ID NO.: 39, SEQ. ID NO.: 41, and pharmaceutical equivalents thereof. In some embodiments, at least a portion of a sequence of the chimeric protein has an amino acid sequence selected from the group consisting of: SEQ. ID NO.: 40, SEQ. ID NO.: 42, and pharmaceutical equivalents thereof.

In some embodiments, the present disclosure is directed to a method of making a chimeric protein for use in the treatment of a condition, the method comprising the steps of, cloning a nucleotide sequence into a vector, the nucleotide sequence coding for a chimeric protein comprising dystrophin complexed with a transportation region wherein the transportation region allows for transport of the chimeric protein across a cellular membrane, transfecting the vector into a host cell, proliferating the host cell, and isolating the chimeric protein from the host cell. In some embodiments, the step of isolating the chimeric protein from the host cell includes the step of isolating the chimeric protein from a lysate of the host cell. In some embodiments of the method of making a chimeric protein, the nucleotide sequence comprises a sequence selected from the group consisting of: SEQ. ID NO.: 13, SEQ. ID NO.: 15, SEQ. ID NO.: 17, SEQ. ID NO.: 19, SEQ. ID NO.: 29, SEQ. ID NO.: 31, SEQ. ID NO.: 33, SEQ. ID NO.: 35, SEQ. ID NO.: 37, SEQ. ID NO.: 39, SEQ. ID NO.: 41.

In some embodiments of the method of making a chimeric protein, wherein the nucleotide sequence comprises a sequence selected from the group consisting of: SEQ. ID NO.: 21, SEQ. ID NO.: 23, SEQ. ID NO.: 25, SEQ. ID NO.: 27.

In some embodiments of the method of making a chimeric protein, the medical condition includes a muscular dystrophy, diastrophic dysplasia, malignant melanoma, porphyria, alpha-1 antitrypsin deficiency, Aicardi-Goutieres syndrome, cystic fibrosis, progeria, Marfan syndrome, tuberous sclerosis, adrenoleukodystrophy, and the like.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention will be apparent from the following detailed description taken in conjunction with the accompanying drawings.

FIG. 1 shows a schematic view of a chimeric protein for the treatment of dystrophinopathies consistent with some embodiments of the present disclosure.

FIG. 2 shows an immunostaining of HEK-293 cells transfected with VSV-G only against VSV-G antibody.

FIG. 3 shows an immunostaining of HEK-293 cells transfected with the chimeric dystrophin protein shown in FIG. 1 having VSV-G at the N-terminus against anti-VSV-G antibody.

FIG. 4 shows an immunostaining of DMD patient cells with an anti-dystrophin antibody treated with 0.5 m1 of conditioned media derived from HEK-293 cells transfected with chimeric dystrophin having VSV-G at the N-terminus.

FIG. 5 shows an immunostaining of DMD patient cells with an anti-dystrophin antibody treated with 2.0 m1 of conditioned media derived from HEK-293 cells transfected with chimeric dystrophin having VSV-G at the N-terminus.

FIG. 6 shows a flowchart of a method of treating a subject having a medical condition using the chimeric protein shown in FIG. 1.

FIG. 7 shows a flowchart of a method of making the chimeric protein shown in FIG. 1 for use in the treatment of a condition.

FIG. 8 shows another embodiment of the method of making a chimeric protein for use in the treatment of a condition as shown in FIG. 7.

FIG. 9 shows a flowchart of a method of making a chimeric protein shown in FIG. 1 for use in the treatment of a condition.

### DETAILED DESCRIPTION

The embodiments disclosed by the invention are only examples of the many possible advantageous uses and implementations of the innovative teachings presented herein. In general, statements made in the specification of the present application do not necessarily limit any of the various claimed inventions. Moreover, some statements may apply to some inventive features but not to others. In general, unless otherwise indicated, singular elements may be in plural and vice versa with no loss of generality. In the drawings, like numerals refer to like parts through several views.

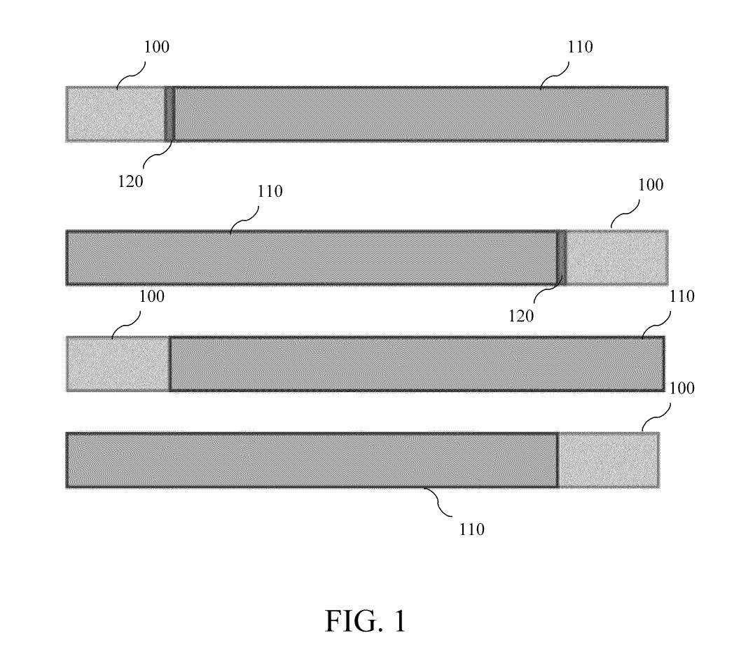

FIG. 1 shows a schematic diagram of a chimeric protein consistent with some embodiments of the present disclosure. In some embodiments, a transportation region is fused to the N-terminal end of a therapeutic region. In some embodiments, the transportation region is fused to the C-terminal end of the therapeutic region. In some embodiments, a cleavage region is disposed between the therapeutic region and the transportation region. As used herein, the term “transportation regions” may also be interchangeably referred to as “delivery mechanisms” or “carriers”. In some embodiments, the transportation region is VSV-G. In some embodiments, the therapeutic region is a full length dystrophin protein. In some embodiments, the therapeutic region is a truncated dystrophin protein. In some embodiments, the cleavage region is a cleavage site for membrane metalloproteases.

In some embodiments, the chimeric protein of the present disclosure has a nucleotide sequence wherein at least a portion of the sequence is selected from the group consisting of: SEQ. ID NO.: 1, SEQ. ID NO.: 3, SEQ. ID NO.: 5, SEQ. ID NO.: 7, and pharmacologically acceptable equivalents thereof. In some embodiments, the chimeric protein of the present disclosure has an amino acid sequence wherein at least a portion of the sequence is selected from the group consisting of: SEQ. ID NO.: 2, SEQ. ID NO.: 4, SEQ. ID NO.: 6, SEQ. ID NO.: 8, and pharmacologically acceptable equivalents thereof. In some embodiments, the sequence for the chimeric protein has at least 90%, at least 95%, at least 96%, at least 97%, at least 98%, or at least 99% sequence identity with at least one of SEQ. ID NO.: 1, SEQ. ID NO.: 2, SEQ. ID NO.: 3, SEQ. ID NO.: 4, SEQ. ID NO.: 5, SEQ. ID NO.: 6, SEQ. ID NO.: 7, SEQ. ID NO.: 8. In some embodiments, any suitable mutations, substitutions, additions, and deletions may be made to the chimeric protein so long as the pharmacological activity of the resulting variant chimeric protein is retained.

SEQ. ID NO.: 1 is a nucleotide sequence of a dystrophin-VSV-G chimeric protein, with the VSV-G at the N-terminus, consistent with some embodiments of the present invention.

SEQ. ID NO.: 2 is an amino acid sequence of a dystrophin-VSV-G chimeric protein, with the VSV-G at the N-terminus, consistent with some embodiments of the present invention.

SEQ. ID NO.: 3 is a nucleotide sequence of a truncated dystrophin-VSV-G chimeric protein, with the VSV-G at the N-terminus, consistent with some embodiments of the present invention.

SEQ. ID NO.: 4 is an amino acid sequence of a truncated dystrophin-VSV-G chimeric protein, with the VSV-G at the N-terminus, consistent with some embodiments of the present invention.

SEQ. ID NO.: 5 is a nucleotide sequence of a dystrophin-VSV-G chimeric protein, with the VSV-G at the C-terminus, consistent with some embodiments of the present invention.

SEQ. ID NO.: 6 is an amino acid sequence of a dystrophin-VSV-G chimeric protein, with the VSV-G at the C-terminus, consistent with some embodiments of the present invention.

SEQ. ID NO.: 7 is a nucleotide sequence of a truncated dystrophin-VSV-G chimeric protein, with the VSV-G at the C-terminus, consistent with some embodiments of the present invention.

SEQ. ID NO.: 8 is an amino acid sequence of a truncated dystrophin-VSV-G chimeric protein, with the VSV-G at the C-terminus, consistent with some embodiments of the present invention.

SEQ. ID NO.: 9 is a nucleotide sequence of a dystrophin-VSV-G chimeric protein without cleavage site with VSV-G at N-terminal, consistent with some embodiments of the present invention.

SEQ. ID NO: 10 is an amino acid sequence of a dystrophin-VSV-G chimeric protein without cleavage site with VSV-G at N-terminal, consistent with some embodiments of the present invention.

SEQ. ID NO: 11 is a nucleotide sequence of a truncated dystrophin-VSV-G chimeric protein without a cleavage site with VSV-G at N-terminal, consistent with some embodiments of the present invention.

SEQ. ID NO: 12 is an amino acid sequence of a truncated dystrophin-VSV-G chimeric protein without a cleavage site with VSV-G at N-terminal, consistent with some embodiments of the present invention.

SEQ. ID NO: 13 is a nucleotide sequence of a penetratin-dystrophin chimeric protein, consistent with some embodiments of the present invention.

SEQ. ID NO: 14 is an amino acid sequence of a penetratin-dystrophin chimeric protein, consistent with some embodiments of the present invention.

SEQ. ID NO: 15 is a nucleotide sequence of a tat-dystrophin chimeric protein, consistent with some embodiments of the present invention.

SEQ. ID NO: 16 is an amino acid sequence of a tat-dystrophin chimeric protein, consistent with some embodiments of the present invention.

SEQ. ID NO: 17 is a nucleotide sequence of a transportan-dystrophin chimeric protein, consistent with some embodiments of the present invention.

SEQ. ID NO: 18 is an amino acid sequence of a transportan-dystrophin chimeric protein, consistent with some embodiments of the present invention.

SEQ. ID NO: 19 is a nucleotide sequence of a TfRbp-dystrophin chimeric protein, consistent with some embodiments of the present invention.

SEQ. ID NO: 20 is an amino acid sequence of a TfRbp-dystrophin chimeric protein, consistent with some embodiments of the present invention.

SEQ. ID NO: 21 is a nucleotide sequence of a penetratin-truncated-dystrophin chimeric protein, consistent with some embodiments of the present invention.

SEQ. ID NO: 22 is an amino acid sequence of a penetratin-truncated-dystrophin chimeric protein, consistent with some embodiments of the present invention.

SEQ. ID NO: 23 is a nucleotide sequence of a tat-truncated-dystrophin chimeric protein, consistent with some embodiments of the present invention.

SEQ. ID NO: 24 is an amino acid sequence of a tat-truncated-dystrophin chimeric protein, consistent with some embodiments of the present invention.

SEQ. ID NO: 25 is a nucleotide sequence of a transportan-truncated-dystrophin chimeric protein, consistent with some embodiments of the present invention.

SEQ. ID NO: 26 is an amino acid sequence of a transportan-truncated-dystrophin chimeric protein, consistent with some embodiments of the present invention.

SEQ. ID NO: 27 is a nucleotide sequence of a TfRbp-truncated-dystrophin chimeric protein, consistent with some embodiments of the present invention.

SEQ. ID NO: 28 is an amino acid sequence of a TfRbp-truncated-dystrophin chimeric protein, consistent with some embodiments of the present invention.

SEQ. ID NO: 29 is a nucleotide sequence of a penetratin-dystrophin-utrophin chimeric protein, consistent with some embodiments of the present invention.

SEQ. ID NO: 30 is an amino acid sequence of a penetratin-dystrophin-utrophin chimeric protein, consistent with some embodiments of the present invention.

SEQ. ID NO: 31 is a nucleotide sequence of a tat-dystrophin-utrophin chimeric protein, consistent with some embodiments of the present invention.

SEQ. ID NO: 32 is an amino acid sequence of a tat-dystrophin-utrophin chimeric protein, consistent with some embodiments of the present invention.

SEQ. ID NO: 33 is a nucleotide sequence of a transportan-dystrophin-utrophin chimeric protein, consistent with some embodiments of the present invention.

SEQ. ID NO: 34 is an amino acid sequence of a transportan-dystrophin-utrophin chimeric protein, consistent with some embodiments of the present invention.

SEQ. ID NO: 35 is a nucleotide sequence of a TfRbp-dystrophin-utrophin chimeric protein, consistent with some embodiments of the present invention.

SEQ. ID NO: 36 is an amino acid sequence of a TfRbp-dystrophin-utrophin chimeric protein, consistent with some embodiments of the present invention.

SEQ. ID NO: 37 is a nucleotide sequence of a dystrophin-utrophin chimeric protein, consistent with some embodiments of the present invention.

SEQ. ID NO: 38 is an amino acid sequence of a dystrophin-utrophin chimeric protein, consistent with some embodiments of the present invention.

SEQ. ID NO: 39 is a nucleotide sequence of a VSV-G-dystrophin-utrophin chimeric protein, consistent with some embodiments of the present invention.

SEQ. ID NO: 40 is an amino acid sequence of a VSV-G-dystrophin-utrophin chimeric protein, consistent with some embodiments of the present invention.

SEQ. ID NO: 41 is a nucleotide sequence of a dystrophin-utrophin-VSV-G chimeric protein, consistent with some embodiments of the present invention.

SEQ. ID NO: 42 is an amino acid sequence of a dystrophin-utrophin-VSV-G chimeric protein, consistent with some embodiments of the present invention.

Recombinant and chimeric proteins have been available in the market as therapies for various conditions and have proven to be extremely potent in curing diseases such as arthritis. In some embodiments, the present disclosure is directed to a dystrophin (or functional mutant or truncated form of dystrophin) protein fused either at the N-terminal or C-terminal with a VSV-G (or variants of VSV-G) protein with or without a short linker between the two proteins, which will be a cleavage site for membrane metalloproteases (MMPs). This therapy will rely on administering biologically effective amounts of recombinant dystrophin protein that will be transduced in different muscle tissues and ameliorate the pathologies associated with the lack of dystrophin. The therapy will allow the subject to compensate for the lack of certain functional protein production.

In some embodiments, VSV-G protein serves as the transportation region or carrier for delivering the dystrophin protein to various tissues. LDL receptors, through which VSV-G establishes initial contact to enter the cells, are present in a wide range of tissues. Therefore, this approach mitigates the issues related to the delivery of dystrophin protein at different target sites found in previous approaches. In some embodiments, the VSV-G used in the chimeric protein of the instant application is a wild-type VSV-G. In some embodiments, the VSV-G is a variant of wild-type VSV-G. Any suitable mutations, substitutions, additions, and deletions may be made to the VSV-G so long as the cellular membrane transport activity of the resulting variant VSV-G is retained. In some embodiments, suitable VSV-G variants include the thermostable and serum resistant mutants of VSV-G, which include the following point mutations to wild-type VSV-G: S162T, T230N, T368A, or combined mutants T230N+T368A, or K66T+S162T+T230N+T368A. In some embodiments, variant VSV-G has at least 90%, at least 95%, at least 96%, at least 97%, at least 98%, or at least 99% sequence identity with wild-type VSV-G.

Sometimes fusing a protein with another protein interferes with the function of the proteins and leads to a decrease in their biological activity. In order to overcome this problem, in some embodiments, a cleavage region including a cleavage site for MMPs is provided. In some embodiments, a cleavable linker is disposed between the dystrophin and VSV-G. In such an embodiment, dystrophin will only be released from the chimeric construct with VSV-G when it is cleaved by MMPs. MMPs are overexpressed during various pathologic conditions and inflammation, including muscular dystrophy. Therefore, when the chimeric dystrophin-VSV-G protein reaches the sites of inflammation upon delivery, the MMPs cleave the chimeric protein and release the dystrophin protein for incorporation into the muscle cells and treatment of the subject's dystrophinopathy. In some embodiments, the MMP cleavage site encodes for amino acids PLGLWAL, which is a known putative cleavage site for all the MMPs. In some embodiments, the MMP cleavage site comprises a sequence defined by the amino acids P-X-X-Hy-(S/T), wherein P identifies proline, X is any residue, Hy is a hydrophobic residue, and S/T may either serine or threonine. In some embodiments, the MMP cleavage site is selected from the sequences PLGLWAL, P-X-X-Hy-(S/T), and variants and mutations thereof. In some embodiments, the cleavage site is eliminated.

This strategy can also be extended to other conditions which are caused by a lack of a single protein by replacing dystrophin with the protein of interest for the respective conditions. In some embodiments, the dystrophin is replaced by another therapeutic construct. In these embodiments, the therapeutic construct would be selected to treat the pathological conditions of other diseases, such as diastrophic dysplasia, malignant melanoma, porphyria, alpha-1 antitrypsin deficiency, Aicardi-Goutieres syndrome, cystic fibrosis, progeria, Marfan syndrome, tuberous sclerosis, adrenoleukodystrophy, and the like. In some embodiments, other proteins may be fused to the N- and C-termini of the proteins included in the chimeric protein. Table 1 below portrays various conditions that may be effectively treated using the chimeric proteins of the present disclosure, as well as the therapeutic constructs that may, in some embodiments, be substituted for dystrophin.

TABLE 1
Therapeutic constructs for incorporation into the chimeric protein of
the present disclosure for use in the treatment of various conditions.

| Condition | Therapeutic Construct |
|---|---|
| diastrophic dysplasia | SLC26A2. A transmembrane glycoprotein |
| | which is a sulfate transporter and a |
| | member of the solute family. |
| malignant melanoma | p16(INK4a) and the p14(ARF); proteins |
| | encoded by the CDKN2A and PTEN |
| | genes. |
| porphyria | Delta-aminolevulinate dehydratase; 5- |
| | aminolevulinate synthase 2; |
| | coproporphyrinogen oxidase; |
| | ferrochelatase; hydroxymethylbilane |
| | synthase; protoporphyrinogen oxidase; |
| | uroporphyrinogen decarboxylase; |
| | uroporphyrinogen III synthase. |
| | Proteins encoded by the ALAD, ALAS2, |
| | CPOX, FECH, HMBS, PPOX, UROD, or |
| | UROS genes. |
| alpha-1 antitrypsin | Alpha-1 antitrypsin. A plasma protein and |
| deficiency | serine protease inhibitor. |
| Aicardi-Goutieres | Proteins encoded by the TREX1, |
| syndrome | RNASEH2A, RNASEH2B, and |
| | RNASEH2C genes. Mutations in these |
| | genes may result in absent or dysfunctional |
| | nuclease enzymes. |
| | Proteins encoded by the SAMHD1 genes. |
| | These proteins may be involved in immune |
| | response and/or inflammatory processes. |
| cystic fibrosis | Cystic fibrosis transmembrane |
| | conductance regulator. A transmembrane |
| | chloride channel protein. |
| progeria | Lamin A; lamin C. Proteins located at the |
| | nuclear lamina. |
| Marfan syndrome | Fibrillin-1. A large extracellular matrix |
| | protein. |
| tuberous sclerosis | Hamartin; tuberin. Hamartin is a |
| | peripheral membrane protein. Tuberin |
| | associates with hamartin in a cytosolic |
| | complex. |
| adrenoleukodystrophy | Adrenoleukodystrophy protein. A |
| | peroxisome membrane protein. |

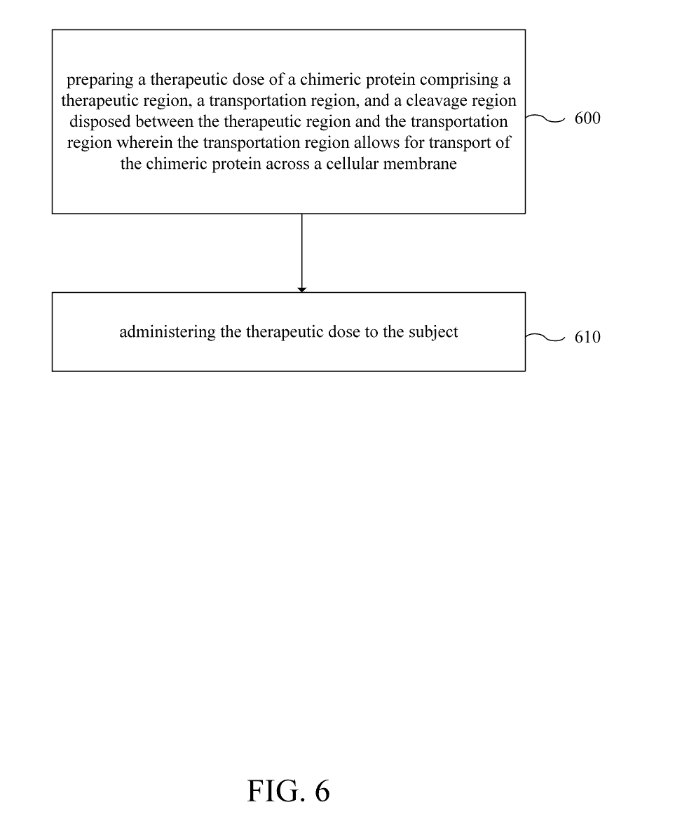

In some embodiments, the present disclosure is also directed to a method of using a chimeric protein consistent with the embodiments described above in the treatment of dystrophinopathies. As shown in FIG. 6, un some embodiments, the method of treating a subject having a dystrophinopathy includes the steps of preparing 600 a therapeutic dose of a chimeric protein comprising a therapeutic region, a transportation region, and a cleavage region disposed between the therapeutic region and the transportation region, wherein the transportation region allows for transport of the chimeric protein across a cellular membrane; and administering 610 the therapeutic dose to the subject. As used herein, the term therapeutic dose means any suitable volume and concentration of the chimeric protein to be administered to a subject as part of a prescribed regimen that is effective for treating a dystrophinopathy. The specific dosage is a matter of design choice and may vary with the characteristics of the subject.

### Methods:

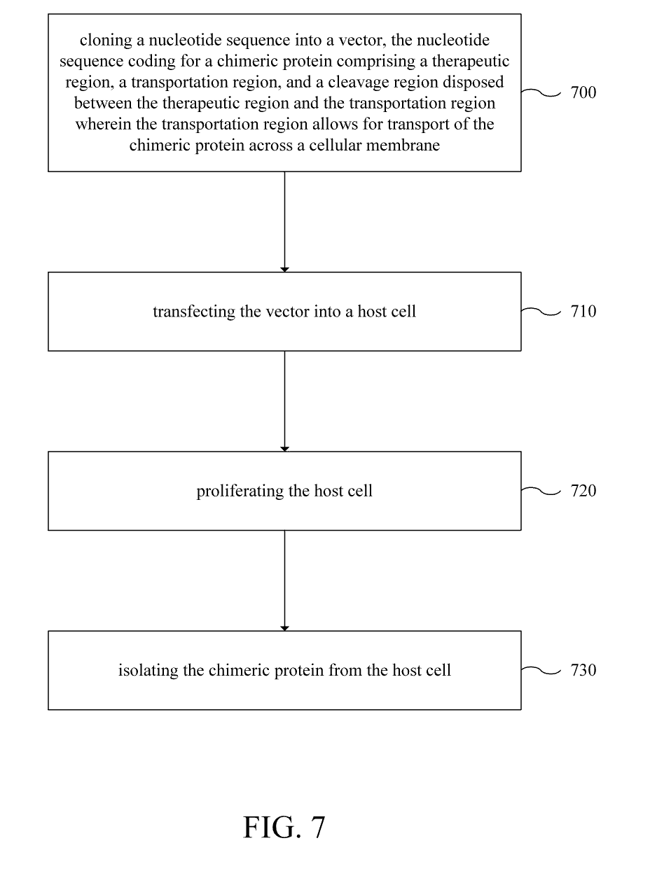

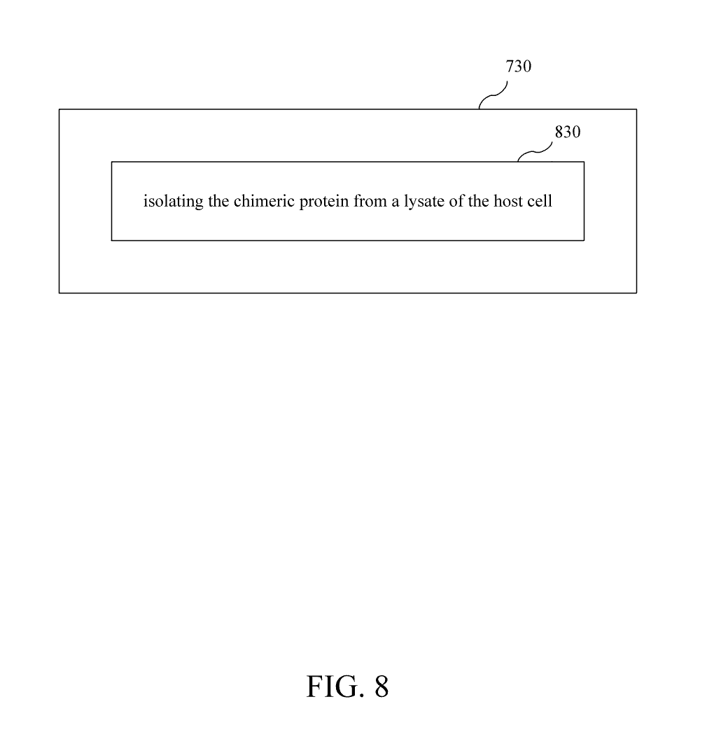

In some embodiments, the present disclosure is also directed to a method of making a chimeric protein consistent with the embodiments described above for use in the treatment of dystrophinopathies. As shown in FIG. 7, in some embodiments, the method of making the chimeric protein includes the steps of cloning 700 a nucleotide sequence into a vector, the nucleotide sequence coding for a chimeric protein comprising a therapeutic region, a transportation region, and a cleavage region disposed between the therapeutic region and the transportation region, wherein the transportation region allows for transport of the chimeric protein across a cellular membrane, transfecting 710 the vector into a host cell, proliferating 720 the host cell, and isolating 730 the chimeric protein from the host cell. In some embodiments, as shown in FIG. 8, isolating step 730 includes the step of isolating 830 the chimeric protein from a lysate of the host cell. Specific examples consistent with some embodiments of the method of making the chimeric protein are as follows:

The dystrophin protein has been described by Kunkel et al., under patent application Ser. No. 07/136,618 and family ID 22473616, the contents of which are incorporated herein by reference in its entirety. The complete mRNA sequence of human dystrophin protein is available at gene bank under accession number M18533, M17154, M18026, and M20250.

The following studies were all performed on a chimeric dystrophin protein without a cleavage region and with VSV-G as the transportation region at the N-terminus of the therapeutic dystrophin.

Plasmid harboring cDNA for full-length Homo Sapiens dystrophin was sourced from Transomic Technologies, 601 Genome Way, Suite 1222, Huntsville, Ala. 35806. Cloning vector pRK-Flag-Myc (Sigma) and pMD2.G (Addgene) harboring the VSV-G cDNA were obtained. The VSV-G protein was cloned at the ApaI and NotI sites of the pRK-Flag-Myc vector thereby replacing the Flag tag with VSV-G. VSV-G protein was cloned by polymerase chain reaction (PCR) using pMD2.G vector as the template and forward and reverse primers as 5′ AAT TAT GGG CCC GAC ACC ATG GAG TGC CTT TTG TAC TTA 3′ and 5′ CTC TAC TTG GCT GAA CCT CGC CGG CGG TTT AGG 3′ respectively.

Next, PCR was performed to amplify the dystrophin open reading frame (ORF) with forward and reverse primers as CCG TCA GCG GCC GCC ATG CTT TGG TGG GAA GAA GTA and TAC TCT CTC CTG TGT TAC CAG CTG GAG TAC respectively, designed to include restriction enzyme sites Not I and Sal I. The dystrophin ORF was cloned at the Not I and Sal I sites of the pRK-Flag-Myc vector following the VSV-G, yielding the chimeric dystrophin with VSV-G at the N-terminal. The KAPA HiFi HotStart ReadyMix PCR kit was used for performing PCR.

Dystrophin and truncated dystrophin chimeric gene sequences with N-terminal VSV-G were synthesized by IDT DNA using their propriety technology and put into appropriate vector for protein expression using a baculovirus system. Methods of protein purification using a baculovirus system are well established and well within the capabilities of one having ordinary skill in the art.

Once the chimeric sequence was generated, it was transfected in mammalian cells to ensure that the chimeric dystrophin protein is being expressed properly. HEK-293 cells were seeded on coverslips in a 6-well plate at a confluence of 60%. Cells were transfected with vectors containing either the VSV-G protein alone or the chimeric dystrophin-VSV-G protein using the Viafect™ reagent from Promega. A day after transfection the media were changed to normal media (10% FBS in DMEM). 48 hours post transfection, the media from the cells was removed.

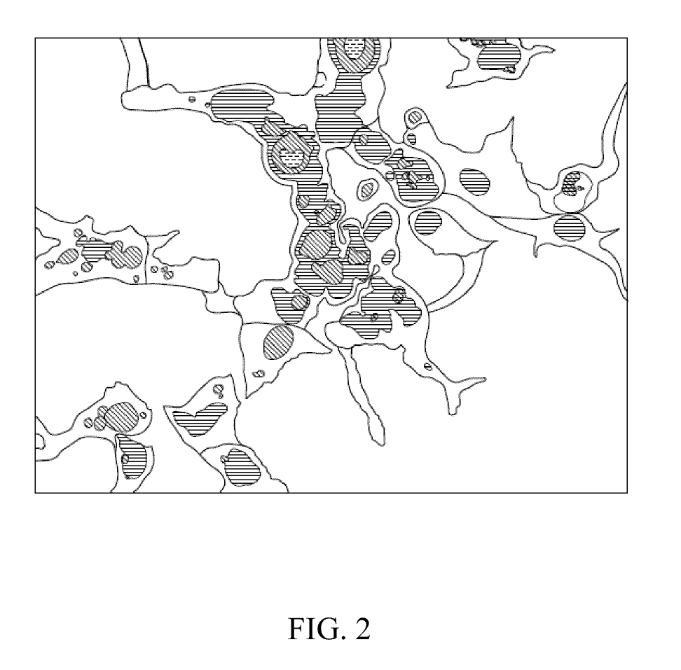

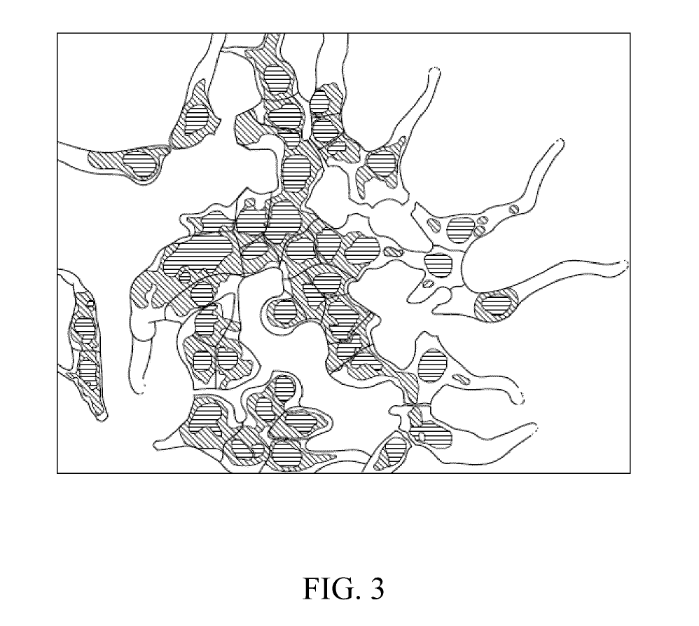

As shown in FIGS. 2 and 3, the transfected cells seeded on coverslips were fixed and stained for detection of dystrophin chimeric protein expression and compared to that of VSV-G alone. For fixation, cells were washed in Dulbecco's phosphate buffered saline (DPBS) and fixed in 4% paraformaldehyde solution for 10 minutes. Cells were washed in DPBS again followed by treatment with 0.5% Triton-X-100 for an additional 5 minutes. Following 3 washes in DPBS, cells were blocked for an hour in 10% BSA. Then primary antibody against VSV-G protein (VSV-G-tag Antibody, pAb, Rabbit, source: GenScript) or dystrophin (monoclonal anti-dystrophin, clone MANDYS8) was added at a dilution of 1:100 in 2.5% BSA-DPBS for one and half hours. Cells were again washed in DPBS. Anti-rabbit FITC conjugated secondary antibody against anti-VSV-G primary or Anti-mouse Texas Red conjugated secondary antibody against anti-dystrophin primary were also added at a dilution of 1:100 in 2.5% BSA-DPBS and incubated for 30 minutes. Cells were washed in DPBS and stained with DAPI in DPBS. Cells were mounted in anti-fade mounting medium.

The stained transfected HEK-293 cells were detected for indirect immunofluorescence under a fluorescence microscope. The transfected cells displayed fluorescence in the cytoplasm confirming the expression of the chimeric protein (FIG. 3). The distribution of the VSV-G protein alone was different from that of the chimeric protein (FIG. 2).

DMD patient cells (GM05169 and GM03604 A) were sourced from Coriell Institute of Biomedical Research. For treatment of DMD patient cells with conditioned media, GM05169 and GM03604 A cells were seeded onto coverslips in 6-well plate. HEK 293 cells were transfected as described above and the media after being changed 24 hours post transfection was left for another 48 hours. 72 hours post transfection, medium from the transfected cells were collected and centrifuged. Different amounts of conditioned HEK transfection media 2.0 ml, 1.0 ml, and 0.5 ml (making a total volume of 2 ml by adding 15% FBS DMEM media) were added to the DMD patient cells and then cells were fixed and stained as described above.

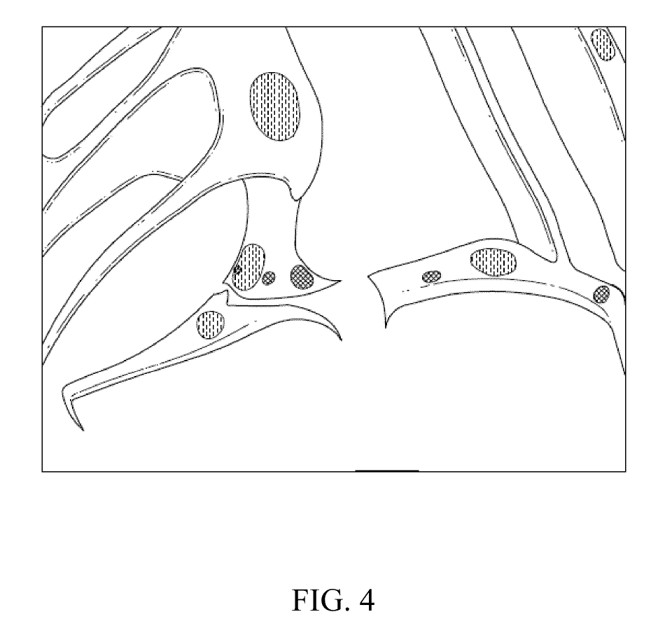

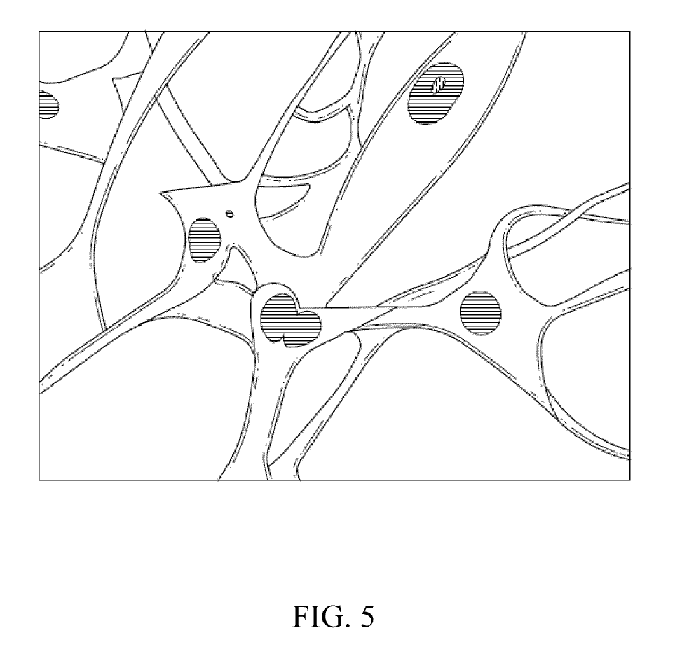

DMD patient cells were then analyzed under a fluorescence microscope for chimeric protein transduction from media to patient cells. As shown in FIGS. 4 and 5, treatment of conditioned transfected HEK-293 cell media indeed resulted in the transduction of chimeric protein that was secreted in the media as displayed by the fluorescence in the cytoplasm of patient cells.

It was postulated that fusion of VSV-G protein with the dystrophin protein would facilitate the entry of the dystrophin protein in the target muscle tissues. VSV-G is a vesicular protein which when overexpressed is secreted in vesicles known as gesicles. It was assumed, therefore, that chimeric dystrophin protein might be secreted as well in the transfection media upon expression. DMD patient cells displaying fluorescence positive for chimeric protein proves that the chimeric protein is secreted in the media of HEK-293 transfected cells and upon treatment of the conditioned media to DMD patient cells, the chimeric protein is taken up by the patient cells.

As discussed above, in some embodiments, chimeric protein expression and functionality is confirmed by inserting DNA encoding the chimeric protein into baculovirus for recombinant protein production. DNA insertion into the baculovirus for recombinant protein production is accomplished using the commercially available pOET1 transfer plasmid (Oxford Expression Technologies) and recombinant baculovirus stocks are prepared for baculovirus-mediated protein expression according to the manufacturer protocols.

Chimeric proteins are produced using recombinant baculovirus stocks to infect suspension cultures of insect cells grown in flasks or bioreactors with commercially available cell lines such as Sf9, Sf21 or Tni and commercially available culture media according to manufacturer protocols. Infected cultures are then harvested between 48 and 96 hours post infection and chimeric proteins are purified from culture media or clarified cell lysate by column chromatography involving one or more methods such as affinity, ion exchange, hydrophobic interaction, and size exclusion.

Purified chimeric proteins are identified by Western blot using one or more dystrophin-specific primary antibodies. Protein purity is determined by densitometry analysis of SDS PAGE stained with SYPRO Orange or Coomassie Blue, and by reverse phase high performance liquid chromatography (RP-HPLC). Protein stability in phosphate buffered saline (PBS) or similar formulation buffer is assessed by analytical size exclusion chromatography (aSEC) to detect changes in protein monodispersity over time and after multiple freeze/thaw cycles. Endotoxin level in purified protein samples is measured by Limulus Amebocyte Lysate (LAL) assay.

Purified chimeric protein produced by the methods described above is administered in mdx mice for further validation of its activity to improve the pathologic effects observed during dystrophinopathies. All the animal studies are done by The Jackson Laboratory.

A variant of mdx mouse in a different background (DBA/2J) is used for the studies. Eighteen hemizygous D2.B10-Dmdmdx/J male mutant mice and six DBA/2J male control mice are produced in the Jackson Laboratory Facility. Three groups of 6 D2.B10-Dmdmdx/J males, age 28 days-3 days, are administered the chimeric protein or vehicle for a six week period. A group of 6 DBA/2J male controls, age 28 days-3 days, are administered just the chimeric protein administration vehicle.

At the end of the treatment period, the following measurements are performed:
Serum Creatine Kinase (CK) activity. Serum CK is measured with a Beckman Coulter AU Clinical Chemistry analyzer from serum collected by retro-orbital bleeding and frozen immediately and until analysis;
Evans Blue Dye (EBD) uptake by the skeletal muscle. EBD is injected 24 hours prior to euthanasia but after serum collection for CK measurement. After euthanasia, the gastrocnemius is collected and flash frozen. Muscles are then homogenized, the lysate is cleared, and EBD concentration in the supernatant is measured by spectrophotometry;
Histology. Hematoxylin and eosin stain for illustration purposes, reticulin stain for muscle fiber morphometry, quantification of the extent of atrophy/hypertrophy and centrally-located nuclei, and Masson Trichrome stain for the visualization of the fibrosis are performed. After sacrifice, one hind limb and one hemi-diaphragm are collected and fixed in paraformaldehyde. Muscles are dissected, paraffin-embedded, and cross-sectioned. Three sections per muscle are prepared to be stained with hematoxylin and eosin for illustration purposes with two mice per group and reticulin stain for automated fiber size measurements and central nuclei counts;
Anti-dystrophin immunofluorescence on the quadriceps, myocardium, and diaphragm. Tibialis anterior and quadriceps muscles are used. One quadriceps and the heart are flash-frozen without fixation for cryo-sectioning and cross-sections are stained by anti-dystrophin immunofluorescence; and
RNA extraction and quantification of the following mRNAs in the tibialis anterior: markers of inflammation, markers of fibrosis, markers of muscle fiber regeneration. One tibialis anterior is preserved in RNA. Later, RNAs are extracted, reverse-transcribed, and the following mRNA quantified by SYBR-Green qPCR: Mpeg1 and Lgals3 (macrophages markers), Ly6c1 (pro-fibrotic monocytes marker), Tnf-alpha (inflammatory cytokine), neonatal myosin (fiber regeneration marker), and collagen I.

In patients suffering from dystroglycanopathies, the function of dystrophin can be replaced by utrophin to some extent. Utrophin, which is otherwise confined to the neuromuscular junctions and myotendinous junctions in the muscles of healthy adult individuals, is over expressed in the muscles of DMD patients and can be found localized along the entire periphery of the muscle fiber. In some embodiments of present application, dystrophin or dystrophin-utrophin chimeric proteins are administered to replace the missing dystrophin protein. In some embodiments, the chimeric protein harbors the N-terminal domain of dystrophin, which is important for binding actin cytoskeleton and C-terminal domain of utrophin that makes association with other members of the DGC.

In some embodiments, the chimeric protein of the present disclosure comprises a dystrophin complexed with a transportation region. In some embodiments, the transportation region is at least one CPP. In some embodiments, the at least one CPP is selected from the group consisting of transcription transactivating protein, penetratin, transportan, transferrin receptor binding peptide, and combinations thereof.

In some embodiments, the chimeric proteins described here are tethered to CPPs at the N-terminal followed by the therapeutic region. In some embodiments, the therapeutic region is a dystrophin, truncated dystrophin, or dystrophin-utrophin chimeric protein. One of the challenges of using chimeric proteins for therapeutic use is the delivery of the proteins at the appropriate target sites. The CPPs enhance the entry of these chimeric proteins at the target sites but less likely to interfere with the structural conformation and consequently function of the core protein due to their short size.

In some embodiments, the chimeric protein includes cell penetrating peptides tethered at the N-terminal of the fully functional dystrophin protein or a truncated functional dystrophin protein. In some embodiments, the chimeric protein includes functional domains from both a dystrophin protein and utrophin. In some embodiments, the chimeric protein includes VSV-G. In some embodiments, the chimeric protein includes both CPPs and VSV-G. In some embodiments, the CPPs or VSV-G are attached to the therapeutic region at the N-terminal or N- and C-terminal r of the dystrophin-utrophin chimeric protein. VSV-G-dystrophin-utrophin fusion protein will facilitate the entry of the protein at the target muscles owing to the presence of LDL receptors in the muscle tissues through which the VSV-G proteins make their entry.

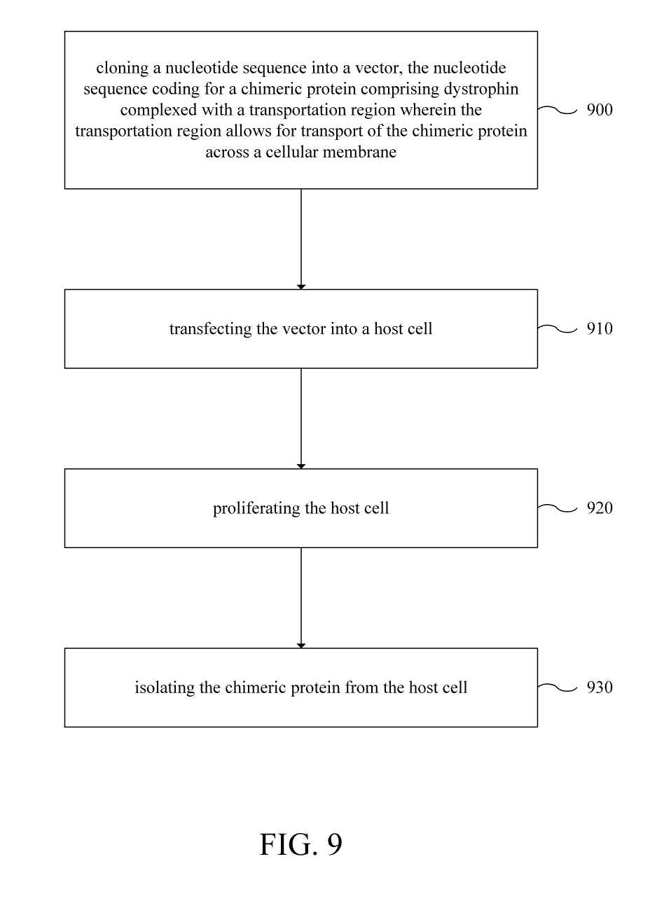

As shown in FIG. 9, in some embodiments, the present disclosure is directed to a method of making a chimeric protein for use in the treatment of a condition, the method comprising the steps of cloning 900 a nucleotide sequence into a vector, the nucleotide sequence coding for a chimeric protein comprising dystrophin complexed with a transportation region wherein the transportation region allows for transport of the chimeric protein across a cellular membrane, transfecting 901 the vector into a host cell, proliferating 902 the host cell, and isolating 903 the chimeric protein from the host cell.

In some embodiments, the chimeric protein of the present disclosure has a nucleotide sequence wherein at least a portion of the sequence is selected from the group consisting of: SEQ. ID NO.: 13, SEQ. ID NO.: 15, SEQ. ID NO.: 17, SEQ. ID NO.: 19, SEQ. ID NO.: 21, SEQ. ID NO.: 23, SEQ. ID NO.: 25, SEQ. ID NO.: 27, SEQ. ID NO.: 29, SEQ. ID NO.: 31, SEQ. ID NO.: 33, SEQ. ID NO.: 35, SEQ. ID NO.: 37, SEQ. ID NO.: 39, SEQ. ID NO.: 41, and pharmacologically acceptable equivalents thereof. In some embodiments, the chimeric protein of the present disclosure has an amino acid sequence wherein at least a portion of the sequence is selected from the group consisting of: SEQ. ID NO.: 14, SEQ. ID NO.: 16, SEQ. ID NO.: 18, SEQ. ID NO.: 20, SEQ. ID NO.: 22, SEQ. ID NO.: 24, SEQ. ID NO.: 26, SEQ. ID NO.: 28, SEQ. ID NO.: 30, SEQ. ID NO.: 32, SEQ. ID NO.: 34, SEQ. ID NO.: 36, SEQ. ID NO.: 38, SEQ. ID NO.: 40, SEQ. ID NO.: 42, and pharmacologically acceptable equivalents thereof. In some embodiments, the sequence for the chimeric protein has at least 90%, at least 95%, at least 96%, at least 97%, at least 98%, or at least 99% sequence identity with at least one of SEQ. ID NO.: 13, SEQ. ID NO.: 14, SEQ. ID NO.: 15, SEQ. ID NO.: 16, SEQ. ID NO.: 17, SEQ. ID NO.: 18, SEQ. ID NO.: 19, SEQ. ID NO.: 20, SEQ. ID NO.: 21, SEQ. ID NO.: 22, SEQ. ID NO.: 23, SEQ. ID NO.: 24, SEQ. ID NO.: 25, SEQ. ID NO.: 26, SEQ. ID NO.: 27, SEQ. ID NO.: 28, SEQ. ID NO.: 29, SEQ. ID NO.: 30, SEQ. ID NO.: 31, SEQ. ID NO.: 32, SEQ. ID NO.: 33, SEQ. ID NO.: 34, SEQ. ID NO.: 35, SEQ. ID NO.: 36, SEQ. ID NO.: 37, SEQ. ID NO.: 38, SEQ. ID NO.: 39, SEQ. ID NO.: 40, SEQ. ID NO.: 41, SEQ. ID NO.: 42.

In some embodiments, any suitable mutations, substitutions, additions, and deletions may be made to the chimeric protein so long as the pharmacological activity of the resulting variant chimeric protein is retained. One or more embodiments of the present invention have been described. Nevertheless, it will be understood that various modifications may be made without departing from the spirit and scope of the invention. Accordingly, other embodiments are within the scope of the following claims.

What is claimed is:

1. A chimeric protein comprising a dystrophin complexed with a transportation region, wherein the transportation region allows for transport of the chimeric protein across a cellular membrane.

2. The chimeric protein according to claim 1, wherein the transportation region is at least one cell penetrating peptide.

3. The chimeric protein of claim 2, wherein said dystrophin is a truncated dystrophin.

4. The chimeric protein of claim 2, wherein said at least one cell penetrating peptide is selected from the group consisting of transcription transactivating protein, penetratin, transportan, transferrin receptor binding peptide, and combinations thereof.

5. The chimeric protein of claim 2, wherein said at least one cell penetrating peptide is attached to either the N-terminal or the N- and the C-terminal.

6. The chimeric protein of claim 5, wherein said at least one cell penetrating peptide is complexed at the N-terminal of the dystrophin.

7. The chimeric protein of claim 2, wherein at least a portion of a sequence of the chimeric protein has a nucleotide sequence selected from the group consisting of: SEQ. ID NO.: 13, SEQ. ID NO.: 15, SEQ. ID NO.: 17, SEQ. ID NO.: 19, and pharmaceutical equivalents thereof.

8. The chimeric protein of claim 2, wherein at least a portion of a sequence of the chimeric protein has an amino acid sequence selected from the group consisting of: SEQ. ID NO.: 14, SEQ. ID NO.: 16, SEQ. ID NO.: 18, SEQ. ID NO.: 20, and pharmaceutical equivalents thereof.

9. The chimeric protein of claim 3, wherein at least a portion of a sequence of the chimeric protein has a nucleotide sequence selected from the group consisting of: SEQ. ID NO.: 21, SEQ. ID NO.: 23, SEQ. ID NO.: 25, SEQ. ID NO.: 27, and pharmaceutical equivalents thereof.

10. The chimeric protein of claim 3, wherein at least a portion of a sequence of the chimeric protein has an amino acid sequence selected from the group consisting of: SEQ. ID NO.: 22, SEQ. ID NO.: 24, SEQ. ID NO.: 26, SEQ. ID NO.: 28, and pharmaceutical equivalents thereof.

11. The chimeric protein of claim 1, further comprising a utrophin complexed with said dystrophin.

12. The chimeric protein of claim 11, wherein at least a portion of a sequence of the chimeric protein has a nucleotide sequence selected from the group consisting of: SEQ. ID NO.: 29, SEQ. ID NO.: 31, SEQ. ID NO.: 33, SEQ. ID NO.: 35, SEQ. ID NO.: 37, and pharmaceutical equivalents thereof.

13. The chimeric protein of claim 8, wherein at least a portion of a sequence of the chimeric protein has an amino acid sequence selected from the group consisting of: SEQ. ID NO.: 30, SEQ. ID NO.: 32, SEQ. ID NO.: 34, SEQ. ID NO.: 36, SEQ. ID NO.: 38, and pharmaceutical equivalents thereof.

14. The chimeric protein of claim 1, wherein the transportation region is vesicular stomatitis virus G.

15. The chimeric protein of claim 14, further comprising a utrophin complexed with said dystrophin.

16. The chimeric protein of claim 15, wherein at least a portion of a sequence of the chimeric protein has a nucleotide sequence selected from the group consisting of: SEQ. ID NO.: 39, SEQ. ID NO.: 41, and pharmaceutical equivalents thereof.

17. The chimeric protein of claim 15, wherein at least a portion of a sequence of the chimeric protein has an amino acid sequence selected from the group consisting of: SEQ. ID NO.: 40, SEQ. ID NO.: 42, and pharmaceutical equivalents thereof.

18. A method of making a chimeric protein for use in the treatment of a condition, the method comprising the steps of:
cloning a nucleotide sequence into a vector, the nucleotide sequence coding for a chimeric protein comprising dystrophin complexed with a transportation region wherein the transportation region allows for transport of the chimeric protein across a cellular membrane;
transfecting the vector into a host cell;
proliferating the host cell; and
isolating the chimeric protein from the host cell.

19. The method of making a chimeric protein according to claim 18, wherein the nucleotide sequence comprises a sequence selected from the group consisting of: SEQ. ID NO.: 13, SEQ. ID NO.: 15, SEQ. ID NO.: 17, SEQ. ID NO.: 19, SEQ. ID NO.: 29, SEQ. ID NO.: 31, SEQ. ID NO.: 33, SEQ. ID NO.: 35, SEQ. ID NO.: 37, SEQ. ID NO.: 39, SEQ. ID NO.: 41.

20. The method of making a chimeric protein according to claim 18, wherein the dystrophin is a truncated dystrophin.

21. The method of making a chimeric protein according to claim 20, wherein the nucleotide sequence comprises a sequence selected from the group consisting of: SEQ. ID NO.: 21, SEQ. ID NO.: 23, SEQ. ID NO.: 25, SEQ. ID NO.: 27.

22. The method of making a chimeric protein for use in the treatment of a condition according to claim 18, wherein the medical condition includes a muscular dystrophy, diastrophic dysplasia, malignant melanoma, porphyria, alpha-1 antitrypsin deficiency, Aicardi-Goutieres syndrome, cystic fibrosis, progeria, Marfan syndrome, tuberous sclerosis, adrenoleukodystrophy, and the like.

23. The method of making a chimeric protein for use in the treatment of a condition according to claim 18, wherein the step of isolating the chimeric protein from the host cell includes the step of isolating the chimeric protein from a lysate of the host cell.
